(19) United States
(12) Patent Application Publication
(10) Pub. No.: US 2003/0224354 A1
(43) Pub. Date: Dec. 4, 2003

(54) Quantifying viral particles with intrinsic fluorescence

(75) Inventors: Shawn Gallagher, Missouri City, TX (US); Richard Sublett, The Woodlands, TX (US)

Correspondence Address: MARSHALL, GERSTEIN & BORUN LLP, 6300 SEARS TOWER, 233 S. WACKER DRIVE, CHICAGO, IL, 60606, US

(73) Assignee: INTROGEN THERAPEUTICS INC., Austin, TX (US)

(21) Appl. No.: 10/447,809

(22) Filed: May 29, 2003

Related U.S. Application Data
(60) Provisional application No. 60/384,285, filed on May 30, 2002.

(51) Int. Cl. C12Q 1/70; C12Q 1/68

(52) U.S. Cl. 435/5; 435/6

(57) ABSTRACT

The invention relates to methods for quantifying viral particles in preparations by detecting the intrinsic fluorescence of viral proteins and correlating this emission to a standard for that virus.

### BACKGROUND OF THE INVENTION

This application claims the benefit of U.S. Provisional Patent Application Serial No. 60/384,285 filed May 30, 2002.

The present invention relates to methods of quantifying viral particles in compositions such as crude and purified cell preparations. Accurate quantification of viral particles in a given preparation is an important process parameter, often used to monitor production parameters and to calculate the yield and titer of preparations developed for therapeutic applications. The treatment of disease by gene therapy is one such application where therapeutic genes are delivered to diseased cells by viral vectors. Often, the methods used to quantify viral particles in final purified preparations are not adequate to enable the quantification of viral particles in impure samples from early in the production process because of sample impurities and relatively low viral concentration. There is a need for a more accurate method of quantifying viral vector particles.

Several methods exist to quantify purified viral particles but these methods are insufficient for quantifying both crude and purified cell preparations. The classic method for such quantification is plaque assaying using dilution and plating methods. This method is not suitable for quantification during the industrial production processes as it is time consuming to administer, and provides poor reproducibility. Also the viral particle to plaque titer ratio may vary with different viral constructs and in general may range from a ratio of 10 to 100 making plaque titer an imprecise measurement of viral particle concentration.

Radioactivity has also been used to identify viral particles. This method requires labeling a precursor to the virus with a radioactive component. Radiation is not a particularly useful method for in-process quantification as samples of the final purified virus are compared with the level of radioactivity detected in a known standard and extrapolated back to determine the in-process quantity. In addition to the destructive nature of the testing, disposal, cost and product safety concerns are other detriments to using radiation.

Viral particle concentration has been determined by measuring the ultraviolet (UV) absorbance of preparations at certain wavelengths. The absorbance of the test sample is measured, typically at 260 nm, and compared with the absorbance ratio standard for the sample at two wavelengths, one attributable to the protein (280 nm) and another to the DNA (260 nm) at known concentrations. This method of quantification only works in the absence of contaminating materials, such as host cell DNA which also absorbs at the wavelength used to quantify the number of viral particles in the sample., This absorbance methodology is best used after viral purification, such as high performance liquid chromatography (HPLC) because the baseline separation from the DNA increases the accuracy of quantifying the DNA solely attributable to the sample. Even with HPLC, the background signal from the contaminants prevents distinguishing between degraded components and viral particles in the crude preparation using UV spectroscopy.

Fluorescence occurs when radiant light energy boosts an electron in the fluorochrome molecule to a higher energy shell (an unstable, excited state) such that when the excited electron falls back to the ground state, the fluorochrome is shifted towards a longer wavelength (lower energy) compared to the excitation spectrum. The difference in wavelength between the apex of the excitation and emission spectra of a fluorochrome is called the Stokes shift. The intensity of this fluorescence (emittance) is measured perpendicularly to the direction of irradiation and is proportional to the concentration of the fluorescent substance. Thus, fluorescence spectra have utility in both qualitative and quantitative analyses.

Single-molecule fluorescence microscopy and spectroscopy have provided novel insights into the dynamics of complex heterogeneous systems (Nguyen, et al., Anal. Chem. 59:2158-2161 (1987); Mets, et al., J. Fluoresc. 4:259-264 (1994); Nie, et al., Science 266:1018-1021 (1994); Basché, et al., Single-Molecule Optical Detection, Imaging and Spectroscopy (VCH, Weinheim, Germany (1997)); Deniz, et al., Proc. Natl. Acad. Sci. USA 96:3670-3675 (1999); Neuhauser, et al., Phys. Rev. Lett. 85:3301-3304 (2000); Yu, et al., Science 289:1327-1330 (2000)). In the biological sciences, confocal scanning and wide-field fluorescence microscopy of single protein molecules have been used to study conformational transitions (Wennmalm, et al., Proc. Natl. Acad. Sci. USA 94:10641-10646 (1997); Eggeling, et al., Proc. Natl. Acad. Sci. USA 95:1556-1561 (1998); Ha, et al., Proc. Natl. Acad. Sci. USA 96:893-898 (1999); and Chapeaurouge, et al., J. Biol. Chem. 276:14861-14866 (2001)), enzyme kinetics (Lu et al., Science 282:1877-1882 (1998)), local pH in cells (Haupts, et al., Proc. Natl. Acad. Sci. USA 95:13573-13578 (1998)), and diffusion in membranes (Sonnleitner, et al., Chem. Phys. Lett. 300:221-226 (1999)) and cells (Schwille et al., Biophy. J. 77:2251-2265 (1999)). In addition, protein fusions of a fluorescent protein like green fluorescent protein and a protein or molecule of interest have also been used in high-throughput screening (HTS) assays in cellular systems (Brock, et al., Proc. Natl. Acad. Sci. USA 96:10123-10128 (1998) and Koltermann, et al., Proc. Natl. Acad. Sci. USA 95:1421-1426 (1998)).

The intrinsic fluorescence emission of biomolecules has also been utilized and studied (Peck, et al, Proc. Natl. Acad. Sci. 86:4087-4091 (1989); Dickson, et al., Nature 388:355-358 (1997); Bramble, S., J. Forensic Sci. 41:1038-1041 (1996); Maiti, et al., Science 275:530-532 (1997), Wennmalm, et al., Biol. Chem. 382:393-397 (2001)). The amino acids phenylalanine, tyrosine, and tryptophan with aromatic R groups are intrinsically fluorescent biomolecules, which are excited at direct UV wavelengths. Direct UV excitation is problematic because it leads to low photostability of these types of fluorescent molecules (Göppert-Mayer, M. Ann. Phys. 9:273-295 (1931); Denk, et al., Science 248:73-76 (1990)). As a result, these amino acids have not been studied intensely for their application to intrinsic fluorescence detection. To overcome these problems, recent improvements in equipment, wherein the fluorometers are equipped with excitation filters, dichromatic beam splitters, and barrier filters now allow investigators to develop novel intrinsic fluorescence methods (Bramble, et al. at 1038; and Reshetnyak, et al., Biophysical J. 81:1710-1734 (2001)).

Viral DNA has also been quantified using fluorescent dye. After chromatography, viral preparations are treated with detergents, such as SDS, to strip the viral proteins from the double stranded DNA genomes. A fluorescent dye, such as PicoGreen®, is applied and binds to the DNA. The viral DNA is detectable by measuring the emission from the PicoGreen® bound to the DNA using fluorescent spectrometry (Murakami, et al., Analytical Biochemistry 274:283-288, 285 (1999)). Although this method provides a sensitivity up to 2.6×10⁸ particles/ml, fluorescent dyes bind only to intact double-stranded DNA molecules and as such, this method is not applicable for detecting viral particles in crude cell preparations which may be contaminated with cellular DNA (Id., at 284, 285).

Accordingly, there exists a need in the art for a method of quantifying viral particles quickly, efficiently and accurately in the crude cell preparation. These, and other objects and advantages, as well as additional inventive features, are provided by the present invention and will be apparent from the description of the invention provided herein.

### SUMMARY OF THE INVENTION

The present invention describes a method of quantifying viral particles in a preparation with a nuclease, subjecting the treated preparation to at least one chromatography medium to separate the viral particles from other components of the preparation and analyzing the intrinsic fluorescent emissions of one or more aliquots of the viral particles from the preparation.

The aliquot from the present invention may be subjected to radiant energy at a wavelength selected to stimulate intrinsic fluorescence of viral particles in the preparation. Further, the fluorescence emissions of the viral particles may be monitored for an emission reading. Emission readings may be compared to a standard indicative of the quantity of viral particles to determining the number of viral particles in the preparation from a standard. The use of fluorescence in quantifying viral particles by detection of their protein content is particularly useful as the fluorescent signal is not affected by the presence of nucleic acid.

Nucleases used in these methods are preferably endonucleases, DNases or RNases. The viral supernatant in certain embodiments of the invention undergo concentration and diafiltration prior to being treated with a nuclease.

The preparation may be a crude preparation, purified preparation or therapeutic preparation. The term “crude preparation” means a collection of cells including host cells which contain a vector, preferably a viral vector within a viral particle, wherein the cells are removed from the cultured or growth environment and the cell membranes are disrupted by physiological or chemical means. Crude cell preparations have been harvested and then lysed according to procedures known by one of skill in the art. Crude cell preparations may also be concentrated and diafiltered as that process is performed in the art and/or treated with a nuclease.

The term “purified preparation” means a collection of purified viral cells subjected to at least one chromatography step, aside from any separation, such as HPLC, that might be performed immediately prior to exciting the sample with radiant energy in the fluorometer. Where a single chromatography step is employed to yield the purified preparation, the single-chromatographic step purification methods described by Zhang et al., in U.S. Pat. No. 6,194,191 are preferred and incorporated herein by reference in their entirety.

The term “therapeutic preparation” means a purified preparation combined with a pharmaceutically acceptable carrier for delivery as a therapeutic, including as a gene therapy. The phrases “pharmaceutically or pharmacologically acceptable” refer to molecular entities and compositions that do not produce an adverse, allergic or other untoward reaction when administered to an animal, or a human, as appropriate. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic preparation is contemplated. Supplementary active ingredients also can be incorporated into the preparations. Solutions of the active compounds as free base or pharmacologically acceptable salts can be prepared in water suitably mixed with a surfactant, such as hydroxypropylcellulose. Dispersions also can be prepared in glycerol, liquid polyethylene glycols and mixtures thereof and in oils. Under ordinary conditions of storage and use, these preparations contain a preservative to prevent the growth of microorganisms. A pharmaceutically acceptable composition or therapeutic preparation is one that meets the minimal safety requirements set forth from time to time by the U.S. Food and Administration or other similar governing body regulating pharmaceuticals.

The therapeutic preparation may have one or more of the following properties: a virus titer of between about 1×10⁹ and about 1×10¹³ pfu/ml (preferably between about 1×10¹⁰ and about 2.5×10¹¹); a viral particle concentration of between about 1×10¹⁰ and about 2×10¹³ particle/ml (preferably between about 2×10¹¹ and about 1×10¹³); a particle:pfu ratio of between about 10 and about 60 (preferably between about 20 and about 40); limits of BSA of less than about 50 ng BSA per 1×10¹² viral particles (preferably between about 5 ng and 40 ng of BSA per 1×10¹² viral particles); and/or low concentrations of DNA contamination of between about 50 pg and about 1 ng of contaminating human DNA per 1×10¹² (preferably between about 100 pg and about 500 pg of contaminating human DNA per 1×10¹² viral particle).

The term “viral particle” means all the viral components assembled into a particular viral particle. Examples of viral particles that can be quantified according to the methods of the invention include, without limitation those members of the following viral families: myoviridae, siphoviridae, podoviridae, tectiviridae, corticoviridae, lipothrixviridae, fuselloviridae, poxviridae, unnamed African swine fever-like viruses, iridovirdae, baculoviridae, herpesviridae, adenoviridae, papoviridae, polydanviridae, inoviridae, microviridae, gemininiviridae, circoviridae, parvoviridae, hepadnaviridae, retroviridae, cystoviridae, reoviridae, bimaviridae, totiviridae, partitiviridae, hypoviridae, paramyxoviridae, rhabdoviridae, filoviridae, orthomyxoviridae, bunyaviridae, arenaviridae, leviviridae, picornaviridae, sequiviridae, comoviridae, potyviridae, calciviridae, astroviridae, nodaviridae, tetraviridae, tombusviridae, coronaviridae, flaviviridae, togaviridae, bromoviridae, bamaviridae, deltavirus, viriods.

The viral particles may be retroviral particles from the group of single-stranded RNA viruses characterized by an ability to convert their RNA to double-stranded DNA in infected cells by a process of reverse-transcription (Coffin, 1990). The viral particles may be recombinant viral particles, meaning a cell into which a gene, such as a gene from the adenoviral genome or from another cell, has been introduced. The viral particles may be adenoviral particles, such as type 2 and type 5 adenoviral particles. The viral particles may have an exogenous gene construct that encodes a therapeutic gene, such as those that encode for growth factors, those with angiogenic properties, antigens or tumor suppressor genes, such as p53, wild-type p53, RB, APC, DCC, NF-1, NF-2, WT-1, MEN-I, MEN-II, zac1, p73, BRCA1, VHL, FCC, MMAC1, MCC, p16, p21, p57, C-CAM, p27 and BRCA2.

Chromatography mediums used in these methods preferably provide an environment with a pH of between about 7 to about 10. The chromatography medium may be controlled by computed software and is preferably HPLC.

An embodiment of this invention is a method of quantifying viral particles in a preparation where a cell growth module is seeded with viral producer cells, the viral producer cells are lysed to yield viral supernatant, such supernatant is treated with a nuclease which is subjected to at least one chromatography medium to separate the viral particles from other components of the preparation and the intrinsic fluorescent emission is analyzed from one or more aliquots of the viral particles from the preparation.

In certain embodiments of the invention, the cell growth module is a bioreactor. The cell growth module has an inlet and outlet port that regulate the flow of media. In some embodiments, the viral producer cells are provided nutrients by perfusion.

Another embodiment of the invention is a method of making a therapeutic preparation comprising treating a viral preparation with a nuclease, subjecting the treated viral preparation to at least one chromatography medium to separate the viral particles from other components of the preparation, analyzing the intrinsic fluorescent emissions of one or more aliquots of the viral particles from the preparation, and formulating the preparation to provide a therapeutic preparation.

An embodiment of this invention is treating a patient with a therapeutic preparation prepared by a process including treating a preparation with a nuclease, subjecting the treated preparation to at least one chromatography medium to separate the viral particles from other components of the preparation, analyzing the intrinsic fluorescent emissions of one or more aliquots of the viral particles from the preparation, formulating the preparation to provide a therapeutic preparation and administering said therapeutic preparation to a patient. The patient may be a cancer patient.

Another embodiment of the invention is a method of making a therapeutic preparation comprising seeding a cell growth module with viral producer cells, lysing the viral producer cells to yield viral supernatant; treating the viral supernatant with a nuclease to yield a viral preparation; subjecting the viral preparation to at least one chromatography medium to separate viral particles from other components of the preparation, analyzing the intrinsic fluorescent emission from one or more aliquots of the viral particles from the preparation, formulating the preparation to provide a therapeutic preparation.

An embodiment of this invention is treating a patient with a therapeutic preparation prepared by a process including seeding a cell growth module with viral producer cells, lysing the viral producer cells to yield viral supernatant, treating the viral supernatant with a nuclease, subjecting the viral supernatant to at least one chromatography medium to separate the viral particles from other components of the preparation, analyzing the intrinsic fluorescent emission from one or more aliquots of the viral particles from the preparation, formulating the preparation to provide a therapeutic preparation and administering said therapeutic preparation to a patient.

### DETAILED DESCRIPTION OF THE INVENTION

The present invention addresses the need for a quantification technique capable of detecting viral particles in crude cell preparations. This invention functions without using light spectrometry, radiation or dyes. Methods of the present invention quantify viral particles by detecting their intrinsic fluorescence emissions. Fluorescence detection is advantageous over other techniques, such as radiation and light spectrometry, for viral particle quantification because protein emission signals can be detected with minimal interference from the background noise. As a result, fluorometers detecting protein emissions provide accurate viral particle counts from preparation.

Various processes for the production of viral preparations are known in the art, with a preferred processes comprising that described in U.S. Pat. No. 6,194,191. This process takes advantage of controlled rate perfused cell culture systems, in order to maintain desired levels of certain metabolites and to remove metabolic waste products, thereby reducing impurities in the culture medium. Bioreactors have been widely used for the production of viral particles from both suspension and anchorage dependent animal cell cultures. For example, the most widely used producer cells for adenoviral particle production are anchorage dependent human embryonic kidney cells (293 cells). Bioreactors for vector production such as adenoviral vectors should have the characteristics of high, volume-specific culture surface-area in order to achieve high producer cell density and high virus yield. Microcarrier cell culture in stirred tank bioreactors provides a very high, volume-specific culture surface-area and has been used for the production of viral vaccines (Griffiths, 1986). Furthermore, stirred tank bioreactors have industrially been proven to be scaleable. The Cellcube™ (Coming-Costar) module provides a large styrenic surface area for the immobilization and growth of substrate attached cells. It is an integrally encapsulated sterile single-use device that has a series of parallel culture plates joined to create thin-sealed laminar flow spaces between adjacent plates. The Cellcube™ module has inlet and outlet ports that are diagonally opposite each other and help regulate the flow of media. During the first few days of growth the culture is generally satisfied by the media contained within the system after initial seeding. The amount of time between the initial seeding and the start of the media perfusion is dependent on the density of cells in the seeding inoculum and the cell growth rate. The measurement of nutrient concentration in the circulating media is a good indicator of the status of the culture. When establishing a procedure it may be necessary to monitor the nutrient's composition at a variety of different perfusion rates to determine the most economical and productive operating parameters. Large scale cell culturing is preferably performed using low to medium perfusion rates to improve the yield of purified virus particles. Typically, perfusion is not carried out continuously, but rather, intermittently as certain media components, such as glucose, start to deplete. In the present invention, the glucose concentration in the medium is preferably maintained at a concentration of between about 0.5 g/L and about 3.0 g/L, and more preferably at a concentration of between about 1.0 g/L and 1.5 g/L.

The multiplate Cellcube™ cell culture system also offers a very high, volume-specific culture surface-area. Cells grow on both sides of the culture plates hermetically sealed together in the shape of a compact cube. Unlike stirred tank bioreactors, the Cellcube™ culture unit is disposable. This is very desirable at the early stage production of viral particles that have an intended end use as a clinical product, such as a gene therapy, because of the reduced capital expenditure, quality control and quality assurance costs associated with disposable systems.

Producer cells, such as the 293 cells, support the replication of adenoviral vectors. Other cell lines also support the growth of adenoviruses including, PER.C6 (IntroGene, NL) 911 (IntroGene, NL) and IT293SF. These producer cells are grown in T-flasks followed by expansion in sterile disposable Nunc Cell Factories (CF10). Cell propagation is performed at 37° C. with 10% CO₂ in Dulbecco's Modified Eagle Medium (DMEM) high glucose supplemented with 10% fetal bovine serum. Trypsin/EDTA is used to detach this adherent cell line during expansions. Vials of the working cell bank are thawed and seeded into five T150 flasks. After approximately three days growth these cells are harvested and used to seed fifteen T150 flasks. These are allowed four days growth time before harvesting to seed two CF10s.

The CF10s are seeded by adding the appropriate numbers of 293 cells and culture media to the CF10 units. After a defined number of growth days each CF10 unit is harvested by draining media from the cells and treating with trypsin/EDTA to detach the 293 cell monolayer. Fresh media is added from a connected sterile vented bottle and transferred to the CF10 once cells are detached. The CF10 is agitated to suspend the cells, and the culture is transferred from the CF10 to a sterile vented bottle. Six CF10s are seeded with an appropriate number of the cells harvested from the two CF10s. After a specified growth period these are harvested to seed the four CellCube™ 100 modules (CellCube™ 4×100). Three CF10 units are harvested at a time to seed each side of the CellCube™ 4×100 bioreactor.

Following the initial propagation of the 293 cells in the Cell Factories, further cell mass buildup occurs in the CellCube™ bioreactor. Four CellCube™ 100 modules linked in parallel provide the growth surface of the bioreactor. The CellCube™ 100 module provides a large, stable, styrenic surface area for the immobilization and growth of substrate attached cells. Vertical growth plates surrounded by media allow for attachment to 2 growth surfaces (2 sides) of each plate. The culture media within the system flows from the oxygenator to the circulation pump, and is pumped into and distributed throughout the CellCube™ modules. The media flows from the outlet of the CellCube™ modules back to the oxygenator, where the media is evenly distributed down the inside surface of the glass oxygenator reservoir. The media is continuously refreshed by the gas mixture being supplied to the oxygenator by the system controller. The fluid flow and gas exchange within the oxygenator is carefully controlled to reduce foaming.

The CellCube™ disposable tubing for the oxygenator is initially assembled; then the oxygenator is etched with NaOH etching solution. Etching occurs 1-2 days prior to final assembly and sterilization. The pH and dissolved oxygen probes are calibrated and the oxygenator assembly and tubing is autoclaved. The disposable sterile circulation loop assembly is then attached to the CellCube™ 4×100 modules and oxygenator in a biological safety cabinet.

Media containing bags and bags designated for waste are attached via disposable tubing sets routed through media and waste pumps. Probe lines and gas supplies are attached to the oxygenator from the controller. The media pump is then turned on to fill the bioreactor. The air, oxygen and CO₂ flow rates are set as are upper and lower pH limits.

The CellCube™ 4×100 is set up, with media circulating, up to one week before seeding to test for leaks and visually assess sterility. Once the setup test period is complete, the seeding of cultured cells into the CellCube™ modules takes place. Cells are harvested from three Nunc CF10s to a 2L sterile vented bottle and counted. Each side of the CellCube™ 4×100 bioreactor is seeded at a range of 1.5-3.5×10⁹ total viable cells.

The 2 L sterile vented bottle containing the cells is attached to a sterile 50 L bag that is part of the bioreactor assembly and the correct volume of cells is transferred to the bag. The bottle is swirled during the process to mix the cells evenly. The media in the CellCube™ 4×100 modules is drained into the bag, mixing with the cells. When the CellCube™ 4×100 modules are substantially drained of media, the cell suspension is transferred back into the modules. When the CellCube™ 4×100 modules are full, the module rack is rotated to place the modules on end, allowing the cells to settle and attach to one side of the culture surface. The bioreactor is then incubated for 4-6 hours.

This process is repeated for the second side of the CellCube™ 4×100 modules, seeding at a target value equivalent to the number of cells used to seed the first side. The second side is allowed to incubate 4-18 hours before the modules are returned to the horizontal position and media recirculation is begun.

One day prior to infection, the 10% FBS media container is disconnected and replaced with one containing Dulbecco's Modified Eagle Medium (DMEM) Basal media. This media formulation is fed to the bioreactor for three days (two days post infection) to allow further cell growth while reducing the overall FBS concentration. On day seven or day eight post-seed, three vials of the WVB are thawed to give a Multiplicity of Infection (MOI) of approximately 50 viral particles per cell (approximately 8×10¹⁰ total cells). The material is withdrawn from the vials by syringe, pooled and attached to the bioreactor injection port. Media from the bioreactor is then drawn into the syringe and dispensed back into the bioreactor system with the virus. This draw and dispense process is repeated multiple times to mix the viral suspension and rinse the syringe. The media feed pump is then turned off to prevent WVB dilution and restarted approximately 1 hour after injection to continue feeding. The CellCube™ is then incubated for four to six days. During the incubation period, viral replication and cell lysis occurs. The cells may autolyse, where the cells are left undisturbed until spontaneous lysis occurs; or lysis may be triggered by a standard lysis technique known to those in the art, such as using a solution composed of buffered detergent.

Following the incubation period, the supernatant harvest is recovered from the CellCube™. The bioreactor media (comprising the viral supernatant harvest) is drained into the 50 liter bag that is part of the bioreactor assembly. Samples are taken for Quality Control testing before the harvest is passed through a prepared Supernatant Clarification Assembly (5.0 and 0.5 micron filters) into a new 50 liter sterile disposable bag. DMEM basal media is then flushed through the filters and into the bag to increase recovery.

After the supernatant harvest containing adenoviral material is clarified, it undergoes concentration and diafiltration in a 25 square foot 300 KD Pellicon Tangential Flow filtration assembly (Pellicon) that can employ a software controlled Millipore Proflux A60 filtration skid that integrates the Pellicon with a 26 Liter reservoir and associated piping. The Pellicon is tested for integrity and flux rate, sanitized, and rinsed prior to equilibration with basal media. The sterile bag containing the supernatant harvest is then aseptically connected to the system feed pump, which is attached to the Pellicon system. The supernatant harvest is pumped into the reservoir as the material is processed through the Pellicon. An approximate ten-fold concentration is achieved. The buffer is then exchanged with at least 4 times the concentrated sample volume of Diafilter Buffer (0.5M TRIS, pH 8.0, 1 nM MgCl₂). The reservoir containing the product in Diafilter Buffer is drained into a sterile bag, then the Pellicon filter is post-washed with Diafilter Buffer to increase recovery.

The concentrated/diafiltered crude preparation is treated with 100±10 u/mL of Benzonase® (EM Industries, Hawthorne, N.Y.). Nucleases, such as Benzonase® selectively degrade un-encapsulated DNA and RNA without disrupting the recombinant viral vectors. Other preferred nucleases include combinations of endonucleases, DNases and RNases such as: Pulmozyme®, RNase A, T1, RNase I, micrococcal nuclease, S1 nuclease and mung bean nuclease. Nuclease use is advantageous because it reduces agglomeration of nucleic acids to the viral protein coat which interferes with separation. Since nucleic acids do not have an intrinsic fluorescence activity, the use of a nuclease may be desirable to improve elution without affecting intrinsic fluorescence. The crude preparation is filtered with a 0.2 micron filtered. The filter is flushed with Diafilter Buffer to increase recovery. The Benzonas® ™ treated solution is incubated at room temperature in a biological safety cabinet for 18±3 hours. The material is then 0.2 micron filtered in preparation for chromatographic purification. The 0.2 micron filter is flushed with Diafilter Buffer to increase recovery. The filtered adenoviral material may be stored up to 24 hours at 2-8° C. prior to purification.

Anion-exchange chromatographic purification and separation are performed on the adenoviral material using a Pharmacia Bioprocess Purification System (automated chromatographic skid with associated computer controls). Chromatographic purification techniques are well known in the art. These techniques employ membranes known in the art to separate the cellular debris (i.e. protein, complex lipids, and nucleic acid monomers, oligomers etc) from the viral particles of the crude cell preparation, such as size exclusion. Chromatography media such as the following, are well known in the art: affinity mediums, gel filtration, hydrophobic and hydroxyapatite, with preferred chromatography media including anion exchange and High Performance Liquid Chromatography (HPLC). HPLC is characterized by a very rapid separation with extraordinary resolution of peaks. Separation can be achieved in a matter of minutes or at most an hour. Moreover, only a very small volume of the sample is needed to count, for example, virus particles in a particular sample because the void volume is very small volume of the column due to the closely packed resin. Also, the concentration of the sample needed is small due to very little dilution of the sample for application to the beads. Examples of suitable resins include Fractoge.R™ (E. Merck, Gibbstown, N.J.) resins derived from either DEAA or DMAE; Fractogel.R™.EMD Tentacle resin derived from with DEAE, DMAE, or TMAE; Toyopearl.R™. (TasoHaas, Montgomeryville, Pa.) resins derived with DEAA or QAE; Acti-Disk.R™. (Whatman, Clifton, N.J.) supports derived from Quat, DEAE, or PEI; Sepharose.R™. (Pharmacia, Piscataway, N.J.) resins derived from DEAE; Sephacel.R™. (Pharmacia, Piscataway, N.J.) resins derived from DEAE; and Sephadex. R™ resins derived from DEAE and QAE. Preferred anion exchange resins are derived from the DEAE group, and further preferred columns are the Fractogel, Toyopearl, and Streamline.™ (Pharmacia, Piscataway, N.J.) DEAE resins.

Once the computer software is loaded, pH calibration and system checks are performed. The Source 15 Q resin column (Pharmacia, Piscataway, N.J.) and solutions are connected. The column is HETP tested and the system sanitized the day prior to actual purification. The adenoviral purification program is run on the system. Waste bags are monitored throughout the procedure and changed as they fill; buffer containers are changed as needed. When conditioning and equilibration are complete, the load solution is connected to the “sample” inlet port and the line is primed. The sample loading step then occurs. After a column wash step the linear gradient column elution takes place. The outlet changes to product collection with the appearance of the viral peak at the appropriate conductivity and when the UV absorbance at A₂₈₀ rises above 0.1 AU. Collection stops when the peak lowers to 0.2 AU. After product collection is complete, post product eluate and salt strip are collected. Base and acid washing/regeneration procedures follow before the column and system are filled with a dilute NaOH storage solution. Samples are drawn for Quality Control testing, a report is generated and the system is shut down.

After the addition of glycerol (approximately 10% by volume) the column eluate may either be further processed through final vialing, or 0.2 micron filtered into a sterile disposable bag and frozen at ≦−60° C. for up to three months before use.

Final concentration, diafiltration, dilution and filtration of the purified adenoviral preparation is carried out after the column eluate is thawed overnight at room temperature. Concentration and diafiltration is accomplished by the use of a 3.3 square foot 300 KD mini Pellicon Tangential Flow filtration assembly (Pellicon). This Pellicon Assembly is a semi-automated Millipore Proflux M12 filtration unit with 3-Liter removable reservoir and associated piping. The Pellicon is tested, sanitized, and rinsed prior to equilibration with Formulation Buffer (Dulbecco's Phosphate Buffered Saline with 10% glycerol, formulated with bottled water for injection). The sterile bag containing the column eluate is then aseptically connected to the system feed pump, which is attached to the Pellicon system. The column eluate is pumped into the reservoir as the material is processed through the Pellicon. Once feed is completed, a sterile disposable bag containing Formulation Buffer is then aseptically connected and diafiltration is performed until at least 9 times the volume of the concentrated sample is collected in the waste container. The reservoir containing the product in Formulation Buffer is drained into a sterile bag, then the Pellicon filter is post-washed with Formulation Buffer to increase recovery. Samples are then taken for particle enumeration to determine the dilution necessary to reach the desired final formulation concentration (1E12 vp/mL for 10-00007). Aliquots of the product preparation are diluted to the desired viral particle concentration with Formulation Buffer. The final preparation is filtered through a 0.22 micron MilliPak assembly into a sterile disposable bag and labeled appropriately. The bulk product preparation may be stored refrigerated for up to 24 hours. The purified preparation may be placed in a pharmaceutically acceptable composition for delivery as a therapeutic, including as a gene therapy. The ability to produce infectious viral vectors, such as the therapeutic preparation herein, is increasingly important to the pharmaceutical industry as therapies, vaccines and protein production machines, especially in the context of gene therapy. Product is filled into contract-prepared sterile glass vials with stoppers. The stoppered vials are supplied in stainless steel racks of approximately 200 vials each. In order to fill the vials, a sterile destoppering/stoppering device is used to remove and hold the stopper. The same stopper is aseptically reinserted into the vial after filling.

At various points throughout the process it is desirable to sample the crude cell lysate and quantify the viral particles in the preparation. For instance, the crude cell preparation may be sampled post-harvest and lysis, after concentration and diafiltration and post-nuclease treatment. In addition, the purified adenovirus adenovirus may be quantified.

The present invention employs a fluorescence detector to quantify the viral particles in a sample by measuring the intrinsic fluorescence emission reading from the proteins, specifically fluorophores located within the protein of the viral particles. A “fluorochrome” or “fluorophore” is any inorganic or organic substance when irradiated with radiant energy of sufficient intensity and appropriate wavelength absorb energy and these excited molecules immediately emit radiant energy of a longer wavelength. Aromatic amino acids, such as tryptophan, are examples of intrinsic biological fluorophores and are well known in the art.

Due to the abundant presence of aromatic amino acids such as tryptophan, phenylalanine, and tyrosine in viral proteins, the quantification method of the invention does not require the use of any fluorescent dyes, such as PicoGreen®, to detect the viral particles. In fact, the addition of dyes to the viral particles may produce unknown conformational effects to the viral coat proteins (Lippitz, et al., Proc. Natl. Acad. Sci. 99:2772-2777 (2002)). Moreover, the use of fluorescent dyes requires disruption of the virus in order to enable the dye to bind.

Suitable fluorescent detection machines, or fluorometers, for use according to the invention include those such as Waters® 474 Scanning Fluorescence Detector (Millford, Mass.) to measure the intrinsic fluorescence of viral particles in crude cell preparations. In certain aspects of the invention, the fluorometer is coupled with an HPLC column or other separation means to separate the adenoviral particles from contaminants immediately prior to measurement of fluorescence.

Those of ordinary skill in the art are capable of selecting appropriate excitation and emission wavelengths and determining the standard spectra by taking emission readings with known viral particle concentrations. When measuring adenoviral particles, an excitation wavelength of 280 nanometers and an emission wavelength of 325 nanometers are particularly preferred.

The viral particles of the present invention may include classic pharmaceutical preparations for use in therapeutic regimens, including their administration to humans such as for gene therapy. Administration of therapeutic preparations according to the present invention will be via any common route so long as the target tissue is available via that route. This includes oral, nasal, buccal, rectal, vaginal or topical. Alternatively, administration will be by orthotopic, intradermal, subcutaneous, intramuscular, intraperitoneal, or intravenous injection. Such preparations would normally be administered as pharmaceutically acceptable compositions that include physiologically acceptable carriers, buffers or other excipients. For application against tumors, direct intratumoral injection, injection of a resected tumor bed, regional (i.e., lymphatic) or general administration is contemplated. It also may be desired to perform continuous perfusion over hours or days via a catheter to a disease site, e.g., a tumor or tumor site.

The therapeutic preparations of the present invention are advantageously administered in the form of injectable compositions either as liquid solutions or suspensions; solid forms suitable for solution in, or suspension in, liquid prior to injection may also be prepared. These preparations also may be emulsified. A typical composition for such purpose comprises a pharmaceutically acceptable carrier. For instance, the composition may contain about 5 mg of human serum albumin per milliliter of phosphate buffered saline. Other pharmaceutically acceptable carriers including aqueous solutions, non-toxic excipients, salts, preservatives, buffers and the like may be used. Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose and the like. Intravenous vehicles include fluid and nutrient replenishers. Preservatives include antimicrobial agents, anti-oxidants, chelating agents and inert gases. The pH and exact concentration of the various components the pharmaceutical composition are adjusted according to well known parameters.

Additional therapeutic preparations are suitable for oral administration. Oral preparation formulations include such typical excipients as, for example, pharmaceutical grades of mannitol, lactose, starch, magnesium stearate, sodium saccharine, cellulose, magnesium carbonate and the like. The preparations take the form of solutions, suspensions, tablets, pills, capsules, sustained release formulations or powders. When the route of administration is topical, the preparation's form may be a cream, ointment, salve or spray.

An effective amount of the therapeutic agent is determined based on the intended goal, for example (i) inhibition of tumor cell proliferation, (ii) elimination or killing of tumor cells, (iii) vaccination, or (iv) gene transfer for long term expression of a therapeutic gene, i.e., gene therapy. The term “unit dose” refers to physically discrete units suitable for use in a subject, each unit containing a predetermined-quantity of the therapeutic composition calculated to produce the desired responses, discussed above, in association with its administration, i.e., the appropriate route and treatment regimen. The quantity to be administered, both according to number of treatments and unit dose, depends on the subject to be treated, the state of the subject and the result desired. Multiple gene therapy regimens are expected, especially for therapeutic preparations including adenovirus.

In certain embodiments of the present invention, therapeutic preparations include an adenoviral vector encoding a tumor suppressor gene used to treat cancer patients. Typical amounts of an adenovirus vector used in gene therapy of cancer is 10³-10¹⁴ PFU/dose, (10³, 10⁴, 10⁵, 10⁶, 10⁷, 10⁸, 10⁹, 10¹⁰, 10¹¹, 10¹², 10¹³, 10¹⁴) wherein the dose may be divided into several injections at different sites within a solid tumor. The treatment regimen also may involve several cycles of administration of the therapeutic preparation over a period of 3-10 weeks. Administration of the vector for longer periods of time from months to years may be necessary for continual therapeutic benefit. In another embodiment of the present invention, the therapeutic preparation includes an adenoviral vector encoding a therapeutic gene that may be used to vaccinate humans or other mammals. Typically, an amount of virus effective to produce the desired effect, in this case, vaccination, would be administered to a human or mammal so that long term expression of the transgene is achieved and a strong host immune response develops. It is contemplated that a series of injections, for example, a primary injection followed by two booster injections, would be sufficient to induce a strong cell-mediated response, whereas high doses of antigen generally induce an antibody-mediated immune response. Precise amounts of the therapeutic preparations also depend on the judgment of the practitioner and are peculiar to each individual.

### EXAMPLE 1

293 cells are grown, expanded, harvested and lysed by processes such as those discussed above, known by those skilled in the art. After lysis, a standard indicative of the quantity of viral particles in the crude preparation is established empirically by methods known in the art. In particular, suitable fluorescence stimulation and detection wavelengths are readily determined for different viruses for different conditions. A sample of 1×10⁷ viral particles/μl is serially diluted to provide a standard optimal fluorescence standard for which to use to quantify samples of an unknown number of viral particles. Each virus solution, either the control or experimental, is subjected diluted to a concentration of approximately 0.015%. A series of emissions spectra are then recorded based upon these samples to create a standard.

Once the relative fluorescence standard is established with the standard dilutions of known viral particles from the crude preparation, quantification of the unknown viral particles in the sample is determined by monitoring the fluorescence emission caused by excitation at selected excitation wavelengths. These emission readings are compared to the standard normalized to 2×10¹¹ particles per 200 microliters of undiluted eluant to determine the quantity of viral particles.

The intrinsic fluorescence of adenoviral particles from the crude preparation is monitored using a Waters® 474 Scanning Fluorescence Detector (Millford, Mass.) coupled with HPLC to separate the preparation prior to measurement of fluorescence. All solutions are prepared in phosphate buffered saline, pH 7.2 and may be diluted. The temperature of the virus samples is maintained at 4° C. until injected. The temperature of the column is maintained at 30° C. The peak generated by the adenoviral protein particles of the injected crude preparation is detected. This peak value is adjusted for the dilution of the sample. The viral particles in the sample are quantified by comparing the normalized fluorescence peak with a standard fluorescence spectra indicative of the number of viral particles.

### EXAMPLE 2

The crude cell lysate preparation may be further purified by concentration and diafiltration. After concentration/diafiltration, a standard indicative of the quantity of viral particles in the crude preparation is established empirically by methods known in the art. In particular, suitable fluorescence stimulation and detection wavelengths are readily determined for different viruses for different conditions. A sample of 1×10⁷ viral particles/μl is serially diluted to provide a standard optimal fluorescence standard for which to use to quantify samples of an unknown number of viral particles. Each virus solution, either the control or experimental, is subjected diluted to a concentration of approximately 0.015%. A series of emissions spectra are then recorded based upon these samples to create a standard.

Once the relative fluorescence standard is established with the standard dilutions of known viral particles from crude cell preparations, quantification of the unknown viral particle sample is determined by monitoring the fluorescence emission caused by excitation at selected excitation wavelengths. These emission readings are compared to the standard normalized to 2×10¹¹ particles of undiluted eluant to determine the quantity of viral particles.

The intrinsic fluorescence of adenoviral particles from the crude preparation is monitored using a Waters® 474 Scanning Fluorescence Detector (Millford, Mass.) coupled with HPLC to separate the preparation prior to measurement of fluorescence. All solutions are prepared in phosphate buffered saline, pH 7.2 and may be diluted. The temperature of the virus samples is maintained at 4° C. until injected. The temperature of the column is maintained at 30° C. The peak generated by the injected crude preparation is detected. This peak value is adjusted for the dilution of the sample. The viral particles in the sample are quantified by comparing the normalized fluorescence peak with a standard fluorescence spectra indicative of the number of viral particles.

### EXAMPLE 3

After concentration and diafiltration, the crude preparation is treated with a nuclease, such as Benzonase™ (EM Industries, Hawthorne, N.Y.). Using the nuclease-treated crude preparation, a standard indicative of the quantity of viral particles in the crude preparation is established empirically by methods known in the art. In particular, suitable fluorescence stimulation and detection wavelengths are readily determined for different viruses for different conditions. A sample of 1×10⁷ viral particles/μl is serially diluted to provide a standard optimal fluorescence standard for which to use to quantify samples of an unknown number of viral particles. Each virus solution, either the control or experimental, is subjected diluted to a concentration of approximately 0.015%. A series of emissions spectra are then recorded based upon these samples to create a standard.

Once the relative fluorescence standard is established with the standard dilutions of known viral particles from crude preparations, quantification of the unknown viral particle sample is determined by monitoring the fluorescence emission caused by excitation at selected excitation wavelengths. These emission readings are compared to the standard normalized to 2×10¹¹ particles of undiluted eluant to determine the quantity of viral particles.

The intrinsic fluorescence of adenoviral particles from the crude preparation is monitored using a Waters® 474 Scanning Fluorescence Detector (Millford, Mass.) coupled with HPLC to separate the preparation prior to measurement of fluorescence. All solutions are prepared in phosphate buffered saline, pH 7.2 and may be diluted. The temperature of the virus samples is maintained at 4° C. until injected. The temperature of the column is maintained at 30° C. The peak generated by the injected crude preparation is detected. This peak value is adjusted for the dilution of the sample. The viral particles in the sample are quantified by comparing the normalized fluorescence peak with a standard fluorescence spectra indicative of the number of viral particles.

### EXAMPLE 4

The crude cell preparation was purified in accordance with the procedures described in the detailed description. Quantification of the unknown viral particles in this purified preparation was determined by monitoring the fluorescence emission caused by excitation at selected excitation wavelengths. These emission readings were compared to the standard normalized to 2×10¹¹ particles of undiluted eluant to determine the quantity of viral particles.

The intrinsic fluorescence of adenoviral particles from the eluant of the purified preparation was monitored using a Waters® 474 Scanning Fluorescence Detector (Millford, Mass.). All solutions were prepared in phosphate buffered saline, pH 7.2 and their dilution factor ranged from a one to two dilution through a one to sixteen dilution. The temperature of the virus samples was maintained at 4° C. until injected. The temperature of the column is maintained at 30° C. Various quantification tests were run on purified preparations immediately after the preparations were eluted through an Resource Q, 1 mL, HPLC anion exchange column (Amersham, Uppsala, Sweden). The purified preparation was subjected to radiant energy at excitation wavelengths between 249 nanometers and 308 nanometers. Emission wavelengths of between 325-402 nanometers were monitored. (See Table 1) The greatest sensitivity was seen with an excitation wavelength of 280 nanometers and measurement of an emission wavelength of 325 nanometers, which resulted in a peak of 2.98×10⁸ with a one to sixteen viral particle dilution from the eluant and 1.25×10¹⁰ viral particles injected correlates to a normalized (peak area times dilution factor) peak value of 4.77×10⁹. The dilution of the viral eluent from this experiment was one to sixteen, which is desirable in light of the decreased testing sample required.

TABLE 1

| Excitation | | | | | Peak Area |
|---|---|---|---|---|---|
| wave- | Emission | | | Viral | Times |
| length | wavelength | | Dilution | Particles | Dilution |
| (nm) | (nm) | Peak Area | Factor | Injected | Factor |
| 249 | 402 | 3.82 × 10⁶ | 2 | 1 × 10¹¹ | 7.64 × 10⁶ |
| 249 | 402 | 3.58 × 10⁶ | 2 | 1 × 10¹¹ | 7.16 × 10⁶ |
| 308 | 352 | 1.91 × 10⁷ | 2 | 1 × 10¹¹ | 3.82 × 10⁷ |
| 308 | 352 | 2.14 × 10⁷ | 2 | 1 × 10¹¹ | 4.28 × 10⁷ |
| 280 | 352 | 4.59 × 10⁷ | 8 | 2.5 × 10¹⁰ | 3.67 × 10⁸ |
| 280 | 352 | 2.22 × 10⁷ | 16 | 1.25 × 10¹⁰ | 3.55 × 10⁹ |
| 280 | 325 | 2.98 × 10⁸ | 16 | 1.25 × 10¹⁰ | 4.77 × 10⁹ |

These fluorescent measurements were compared to absorbance measurements taken on the same sample dilutions measured with an UV Spectrophotometer (Scientific Instruments, MD) at 260 and at 280 nanometers. This comparison suggests that fluorescence may be a less sensitive quantification technique than measuring UV absorption at lower excitation wavelengths such as 249 nanometers., but a substantially more sensitive technique at the excitation wavelength of 280 nanometers particularly with emission measurements of a wavelength of 325 nanometers. Excitation wavelengths of 308 nm were similar or slightly more sensitive than UV absorbance for detection of virus. These results are consistent with the expected intrinsic fluorescent wavelength spectra of the viral proteins.

All the references cited herein are hereby incorporated in their entireties by reference. While the present invention has been described in terms of specific embodiments, it is understood that variations and modifications will occur to those in the art, all of which are intended as aspects of the present invention. Accordingly, only such limitations as appear in the claims should be place on the invention.

What is claimed:

1. A method of quantifying viral particles in a preparation comprising:
(a) treating the preparation with a nuclease;
(b) subjecting the treated preparation to at least one chromatography medium to separate the viral particles from other components of the preparation;
(c) analyzing the intrinsic fluorescent emissions of one or more aliquots of the viral particles from the preparation.

2. The method of claim 1, wherein the aliquot is subjected to radiant energy at a wavelength selected to stimulate the intrinsic fluorescence of the viral particles in the preparation.

3. The method of claim 1, wherein the fluorescence emissions of the viral particles are monitored for an emission reading.

4. The methods of claim 3, wherein the emission reading is compared to a standard indicative of the quantity of viral particles to determine the number of viral particles in the preparation from a standard.

5. The method of claim 1, wherein the viral particles are retroviral particles.

6. The method of claim 1, wherein the viral particles are adenoviral particles.

7. The method of claim 6, wherein the adenoviral particles are type 2 or type 5 adenoviral particles.

8. The method of claim 6, wherein the adenoviral particles are recombinant viral particles comprising an exogenous gene construct encoding a tumor suppressor gene.

9. The method of claim 8, wherein the tumor suppressor gene is a wild-type p53 gene.

10. The method of claim 1, wherein the chromatography medium provides an environment with a pH of between about 7 to about 10.

11. The method of claim 1, wherein the preparation is a crude preparation.

12. The method of claim 1, wherein the preparation is a purified preparation.

13. The method of claim 1, wherein the nuclease is an endonuclease.

14. The method of claim 1, wherein the nuclease is a DNase.

15. The method of claim 1, wherein the nuclease is a RNase.

16. A method of quantifying viral particles in a preparation comprising:
(a) seeding a cell growth module with viral producer cells;
(b) lysing the viral producer cells to yield viral supernatant;
(c) treating the viral supernatant with a nuclease;
(d) subjecting the viral supernatant to at least one chromatography medium to separate the viral particles from other components of the preparation;
(e) analyzing the intrinsic fluorescent emission from one or more aliquots of the viral particles from the preparation.

17. The method of claim 16, wherein the aliquot is subjected to radiant energy at a wavelength selected to stimulate the intrinsic fluorescence of the viral particles in the preparation.

18. The method of claim 16, wherein the fluorescence emissions of the viral particles are monitored for an emission reading.

19. The method of claim 17, wherein the emission reading is compared to a standard indicative of the quantity of viral particles to determine the number of viral particles in the preparation from a standard.

20. The method of claim 16, wherein the chromatography medium is controlled by computer software.

21. The method of claim 16, wherein the chromatography medium is HPLC.

22. The method of claim 16, wherein the cell growth module is a bioreactor.

23. The method of claim 16, wherein the cell growth module has an inlet port and an outlet port.

24. The method of claim 23, wherein the inlet port and the outlet port regulate the flow of media.

25. The method of claim 16, wherein the viral producer cells are provided nutrients by perfusion.

26. The method of claim 16, wherein the viral supernatant undergoes concentration and diafiltration prior to being treated with a nuclease.

27. The method of claim 16, wherein the viral particles are retroviral particles.

28. The method of claim 16, wherein the viral particles are adenoviral particles.

29. The method of claim 28, wherein the adenoviral particles are type 2 or type 5 adenoviral particles.

30. The method of claim 28, wherein the adenoviral particles are recombinant viral particles comprising an exogenous gene construct encoding a tumor suppressor gene.

31. The method of claim 30, wherein the tumor suppressor gene is a wild-type p53 gene.

32. The method of claim 16, wherein the chromatography medium provides an environment with a pH of between about 7 to about 10.

33. The method of claim 16, wherein the preparation is a crude preparation.

34. The method of claim 16, wherein the preparation is a purified preparation.

35. The method of claim 16, wherein the nuclease is an endonuclease.

36. The method of claim 16, wherein the nuclease is a DNase.

37. The method of claim 16, wherein the nuclease is a RNase.

38. A method of treating a patient with a therapeutic preparation comprising:
(a) obtaining a therapeutic preparation that has been prepared by a process including:
(i) treating a preparation with a nuclease;
(ii) subjecting the treated preparation to at least one chromatography medium to separate the viral particles from other components of the preparation;
(iii) analyzing the intrinsic fluorescent emissions of one or more aliquots of the viral particles from the preparation;
(iv) formulating the preparation to provide a therapeutic preparation;
(b) administering said therapeutic preparation to a patient.

39. The method of claim 38, wherein the viral particles are retroviral particles.

40. The method of claim 38, wherein the viral particles are adenoviral particles.

41. The method of claim 40, wherein the adenoviral particles are type 2 or type 5 adenoviral particles.

42. The method of claim 40, wherein the adenoviral particles are recombinant viral particles comprising an exogenous gene construct encoding a tumor suppressor gene.

43. The method of claim 42, wherein the tumor suppressor gene is a wild-type p53 gene.

44. The method of claim 38, wherein the chromatography medium provides an environment with a pH of between about 7 to about 10.

45. The method of claim 38, wherein the preparation is a purified preparation.

46. The method of claim 38, wherein the nuclease is an endonuclease.

47. A method of treating a patient with a therapeutic preparation comprising:
(a) obtaining a therapeutic preparation that has been prepared by a process including:
(i) seeding a cell growth module with viral producer cells;
(ii) lysing the viral producer cells to yield viral supernatant;
(iii) treating the viral supernatant with a nuclease;
(iv) subjecting the viral supernatant to at least one chromatography medium to separate the viral particles from other components of the preparation;
(v) analyzing the intrinsic fluorescent emission from one or more aliquots of the viral particles from the preparation;
(iv) formulating the preparation to provide a therapeutic preparation;
(b) administering said therapeutic preparation to a patient.

48. The method of claim 47, wherein the chromatography medium is controlled by computer software.

49. The method of claim 47, wherein the chromatography medium is HPLC.

50. The method of claim 47, wherein the cell growth module is a bioreactor.

51. The method of claim 47, wherein the cell growth module has an inlet port and an outlet port.

52. The method of claim 51, wherein the inlet port and the outlet port regulate the flow of media.

53. The method of claim 47, wherein the viral producer cells are provided nutrients by perfusion.

54. The method of claim 47, wherein the viral supernatant undergoes concentration and diafiltration prior to being treated with a nuclease.

55. The method of claim 47, wherein the viral particles are retroviral particles.

56. The method of claim 47, wherein the viral particles are adenoviral particles.

57. The method of claim 56, wherein the adenoviral particles are type 2 or type 5 adenoviral particles.

58. The method of claim 56, wherein the adenoviral particles are recombinant viral particles comprising an exogenous gene construct encoding a tumor suppressor gene.

59. The method of claim 58, wherein the tumor suppressor gene is a wild-type p53 gene.

60. The method of claim 47, wherein the chromatography medium provides an environment with a pH of between about 7 to about 10.

61. The method of claim 47, wherein the preparation is a purified preparation.

62. The method of claim 47, wherein the nuclease is an endonuclease.

63. The method for making a therapeutic preparation comprising:
(a) treating a viral preparation with a nuclease;
(b) subjecting the treated viral preparation to at least one chromatography medium to separate the viral particles from other components of the preparation;
(c) analyzing the intrinsic fluorescent emissions of one or more aliquots of the viral particles from the preparation;
(d) formulating the preparation to provide a therapeutic preparation.

64. The method of claim 63, wherein the aliquot is subjected to radiant energy at a wavelength selected to stimulate the intrinsic fluorescence of the viral particles in the preparation.

65. The method of claim 63, wherein the fluorescence emissions of the viral particles are monitored for an emission reading.

66. The methods of claim 65, wherein the emission reading is compared to a standard indicative of the quantity of viral particles to determine the number of viral particles in the preparation from a standard.

67. The method of claim 63, wherein the viral particles are retroviral particles.

68. The method of claim 63, wherein the viral particles are adenoviral particles.

69. The method of claim 68, wherein the adenoviral particles are type 2 or type 5 adenoviral particles.

70. The method of claim 68, wherein the adenoviral particles are recombinant viral particles comprising an exogenous gene construct encoding a tumor suppressor gene.

71. The method of claim 70, wherein the tumor suppressor gene is a wild-type p53 gene.

72. The method of claim 63, wherein the chromatography medium provides an environment with a pH of between about 7 to about 10.

73. The method of claim 63, wherein the preparation is a purified preparation.

74. The method of claim 63, wherein the nuclease is an endonuclease.

75. The method for making a therapeutic preparation comprising:
(a) seeding a cell growth module with viral producer cells;
(b) lysing the viral producer cells to yield viral supernatant;
(c) treating the viral supernatant with a nuclease to yield a viral preparation;
(d) subjecting the viral preparation to at least one chromatography medium to separate viral particles from other components of the preparation;
(e) analyzing the intrinsic fluorescent emission from one or more aliquots of the viral particles from the preparation;
(f) formulating the preparation to provide a therapeutic preparation.

76. The method of claim 75, wherein the aliquot is subjected to radiant energy at a wavelength selected to stimulate the intrinsic fluorescence of the viral particles in the preparation.

77. The method of claim 75, wherein the fluorescence emissions of the viral particles are monitored for an emission reading.

78. The method of claim 77, wherein the emission reading is compared to a standard indicative of the quantity of viral particles to determine the number of viral particles in the preparation from a standard.

79. The method of claim 75, wherein the chromatography medium is controlled by computer software.

80. The method of claim 75, wherein the chromatography medium is HPLC.

81. The method of claim 75, wherein the cell growth module is a bioreactor.

82. The method of claim 75, wherein the cell growth module has an inlet port and an outlet port.

83. The method of claim 82, wherein the inlet port and the outlet port regulate the flow of media.

84. The method of claim 75, wherein the viral producer cells are provided nutrients by perfusion.

85. The method of claim 75, wherein the viral supernatant undergoes concentration and diafiltration prior to being treated with a nuclease.

86. The method of claim 75, wherein the viral particles are retroviral particles.

87. The method of claim 75, wherein the viral particles are adenoviral particles.

88. The method of claim 87, wherein the adenoviral particles are type 2 or type 5 adenoviral particles.

89. The method of claim 87, wherein the adenoviral particles are recombinant viral particles comprising an exogenous gene construct encoding a tumor suppressor gene.

90. The method of claim 89, wherein the tumor suppressor gene is a wild-type p53 gene.

91. The method of claim 75, wherein the chromatography medium provides an environment with a pH of between about 7 to about 10.

92. The method of claim 75, wherein the preparation is a purified preparation.

93. The method of claim 75, wherein the nuclease is an endonuclease.
